(19) United States
(12) Patent Application Publication
(10) Pub. No.: US 2022/0277226 A1
(43) Pub. Date: Sep. 1, 2022

(54) ESTIMATING DEVICE, ESTIMATING METHOD, AND ESTIMATING PROGRAM

(71) Applicant: NIPPON TELEGRAPH AND TELEPHONE CORPORATION, Tokyo (JP)

(72) Inventors: Masahiro KOJIMA, Tokyo (JP); Masami TAKAHASHI, Tokyo (JP); Takeshi KURASHIMA, Tokyo (JP); Tatsushi MATSUBAYASHI, Tokyo (JP); Hiroyuki TODA, Tokyo (JP)

(73) Assignee: NIPPON TELEGRAPH AND TELEPHONE CORPORATION, Tokyo (JP)

(21) Appl. No.: 17/634,587

(22) PCT Filed: Aug. 13, 2019
(86) PCT No.: PCT/JP2019/031845

§ 371 Date: Feb. 11, 2022

(51) Int. Cl. G06N 20/00

(52) U.S. Cl. CPC G06N 20/00

(57) ABSTRACT

An estimation apparatus includes an estimation unit configured to estimate an acceptability tensor corresponding to an activity tensor representing an activity log without a corresponding acceptability log, by using a result of previously performed learning of a relationship between an activity tensor representing an activity log recording activities of a user, and an acceptability tensor representing an acceptability log recording an acceptability for a time change of the activities of the user.

### TECHNICAL FIELD

The disclosed technology relates to an estimation apparatus, an estimation method, and an estimation program.

### BACKGROUND ART

Lifestyle-related diseases are asocial issue and account for about 60% of causes of death and about 30% of medical expenses. It is known that improving lifestyle habits such as eating, exercise, sleep, and alcohol intake is effective in preventing lifestyle-related diseases. In preventing lifestyle-related diseases, it would be important to adjust a schedule that changes daily of a person to an ideal schedule in consideration of the general living conditions of the person.

As such, activities such as eating, exercise, and sleeping, which are considered to be effective in preventing lifestyle-related diseases, are subject to adjustment, and even if only a time for such activities is adjusted, the adjustment does not match the daily lifestyle pattern, and thus, it is difficult to put the adjustment into practice. Focusing on this point, efforts have been made to identify activities that are easy to adjust timewise, among all activities also including activities (such as housework, dinner, and bathing), which are not to be adjusted (see NPL 1).

In NPL 1 mentioned above, in order to identify activities that are easy to adjust timewise, two types of data are collected and analyzed: data recording daily activities (hereinafter, also referred to as an “activity log”); and data recording the acceptability for a time change in a case where the time of an activity is considered as having been possibly changeable (hereinafter, also referred to as an “acceptability log”). According to NPL 1, although there were individual differences, there were users who could, for example, adjust their dinner time, and the analysis results suggest that it may be possible to advance the bedtime by encouraging the users to slightly advance their dinner time so that they can perform their following activities earlier.

### CITATION LIST

### Non Patent Literature

NPL 1: Masami Takahashi, Masahiro Kohjima, Takeshi Kurashima, Tatsushi Matsubayashi, Hiroyuki Toda, “Analysis of daily activities and intervention acceptability toward lifestyle habits improvement”, IEICE Technical Report LOIS 2019-1, 119(17): 1-4, 2019.

### SUMMARY OF THE INVENTION

### Technical Problem

In NPL 1, a subject is asked to input time frames of activities to generate an activity log and an acceptability log. If a technique utilizing an existing activity recognition by a sensor or the like is employed, it is possible to estimate an activity log without asking the subject to input the time frames. However, it is not possible to estimate an acceptability log even by employing the activity recognition. This is because information about the acceptability of a user exists only in the mind of the user, and unlike the activity log, the acceptability log cannot be estimated by a sensor or the like.

Thus, the present disclosure provides an estimation apparatus, an estimation method, and an estimation program for estimating an acceptability log from an activity log without a corresponding acceptability log, by using accumulated data including a set of an activity log and an acceptability log.

### Means for Solving the Problem

A first aspect of the present disclosure is an estimation apparatus including an estimation unit configured to estimate an acceptability tensor for an activity tensor representing an activity log without a corresponding acceptability log, by using a result of previously performed learning of a relationship between an activity tensor representing an activity log recording activities of a user, and an acceptability tensor representing an acceptability log recording an acceptability for a time change of the activities of the user.

Furthermore, a second aspect of the present disclosure is an estimation method using a computer to execute a process, the process including estimating an acceptability tensor for an activity tensor representing an activity log without a corresponding acceptability log, by using a result of previously performed learning of a relationship between an activity tensor representing an activity log recording activities of a user, and an acceptability tensor representing an acceptability log recording an acceptability for a time change of the activities of the user.

Furthermore, a third aspect of the present disclosure is an estimation program causing a computer to execute estimating an acceptability tensor for an activity tensor representing an activity log without a corresponding acceptability log, by using a result of previously performed learning of a relationship between an activity tensor representing an activity log recording activities of a user, and an acceptability tensor representing an acceptability log recording an acceptability for a time change of the activities of the user.

### Effects of the Invention

According to the disclosed technique, it is possible to estimate an acceptability tensor representing an acceptability log, from an activity tensor representing an activity log without a corresponding acceptability log, by using a learning result of a relationship between an activity tensor representing an activity log and an acceptability tensor representing an acceptability log.

### BRIEF DESCRIPTION OF DRAWINGS

FIG. 1 is a block diagram illustrating a hardware configuration of an estimation apparatus according to the present embodiment.

FIG. 2 is a block diagram illustrating a functional component of the estimation apparatus according to the present embodiment.

FIG. 3 is a diagram illustrating an example of an activity log and an acceptability log.

FIG. 4 is a diagram illustrating a state where an activity log is represented as a tensor.

FIG. 5 is a table showing an example of a matrix representing a correspondence relationship for each row between approximation values of elements of an input tensor and approximation values of elements of an output tensor.

FIG. 6 is a diagram illustrating a factorization system.

FIG. 7 is a flowchart illustrating a model parameter learning process of the estimation apparatus.

FIG. 8 is a flowchart illustrating a test data process of the estimation apparatus.

### DESCRIPTION OF EMBODIMENTS

Hereinafter, one example of the embodiments of the disclosed technique will be described with reference to the drawings. In the drawings, the same reference numerals are given to the same or equivalent constituent elements and parts. The dimensional ratios in the drawings are exaggerated for convenience of explanation and may differ from the actual ratios.

In NPL 1 mentioned above, the activity log and the acceptability log are data created on the basis of a self-assessment of a user. In NPL 1 mentioned above, the activity log is created by asking a subject to input time frames of activities performed on a certain day. Furthermore, in NPL 1 mentioned above, the acceptability log is created by asking the subject to input, for an activity performed on that day, “a time frame to which the time of the activity may have been shifted”, and for an activity not performed on that day, “a time frame during which the activity may have been performed”. For example, even if the subject had actually eaten dinner at 19:00, when the subject considered that dinner on that day could have been shifted to any time within a range from “18:00 to 20:00”, the activity “dinner” and the time frame of this activity “18:00 to 20:00” is recorded in the acceptability log.

As described above, it is possible to automatically generate an activity log by using a technique utilizing an existing activity recognition by a sensor or the like. On the other hand, it is difficult to automatically generate an acceptability log unlike the activity log. This is because, as mentioned above, the acceptability for time changes of the activities exists only in the mind of the user.

As described below, the inventors of the present disclosure have contrived a technique for estimating an acceptability log from an activity log without a corresponding acceptability log, by using accumulated data including a set of the activity log and the acceptability log. In the disclosed technique, trends are extracted from accumulated data through learning, and an example of the trends includes “several activities performed in a certain time frame to be recorded in the activity log are likely to be shiftable to an earlier or later time frame, and are often recorded as the acceptability log including the earlier and later time frames”. In the disclosed technique, it is possible to estimate an acceptability log from an activity log without a corresponding acceptability log, by utilizing a learning result.

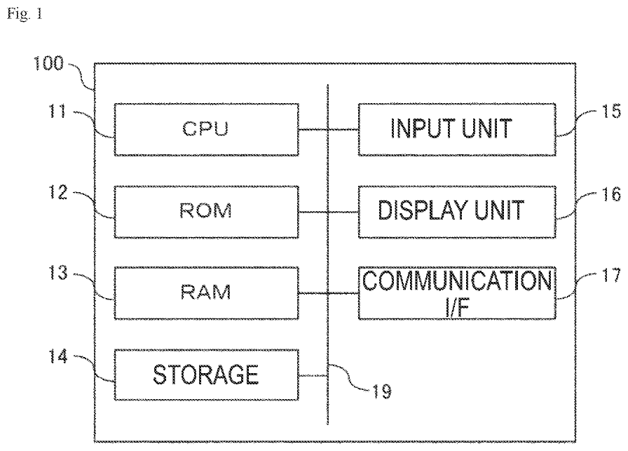

FIG. 1 is a block diagram illustrating a hardware configuration of an estimation apparatus 100.

As illustrated in FIG. 1, the estimation apparatus 100 includes a central processing unit (CPU) 11, a read only memory (ROM) 12, a random access memory (RAM) 13, a storage 14, an input unit 15, a display unit 16, and a communication interface (I/F) 17. The components are communicably interconnected through a bus 19.

The CPU 11 is a central processing unit that executes various programs and controls each unit. In other words, the CPU 11 reads a program from the ROM 12 or the storage 14 and executes the program using the RAM 13 as a work area. The CPU 11 performs control of each of the components described above and various arithmetic processing operations in accordance with a program stored in the ROM 12 or the storage 14. In the present embodiment, the ROM 12 or the storage 14 stores an estimation program for estimating an acceptability log from an activity log without a corresponding acceptability log.

The ROM 12 stores various programs and various kinds of data. The RAM 13 is a work area that temporarily stores a program or data. The storage 14 is constituted by a hard disk drive (HDD) or a solid state drive (SSD) and stores various programs including an operating system and various kinds of data.

The input unit 15 includes a pointing device such as a mouse and a keyboard and is used for performing various inputs.

The display unit 16 is, for example, a liquid crystal display and displays various kinds of information. The display unit 16 may employ a touch panel system and function as the input unit 15.

The communication interface 17 is an interface for communicating with another device such as an external apparatus 200 described later, and uses, for example, a standard such as Ethernet (registered trademark), FDDI, or Wi-Fi (registered trademark).

Next, each functional component of the estimation apparatus 100 will be described. Each functional component is realized by the CPU 11 reading the estimation program stored in the ROM 12 or the storage 14 and loading and executing the estimation program into and in the RAM 13.

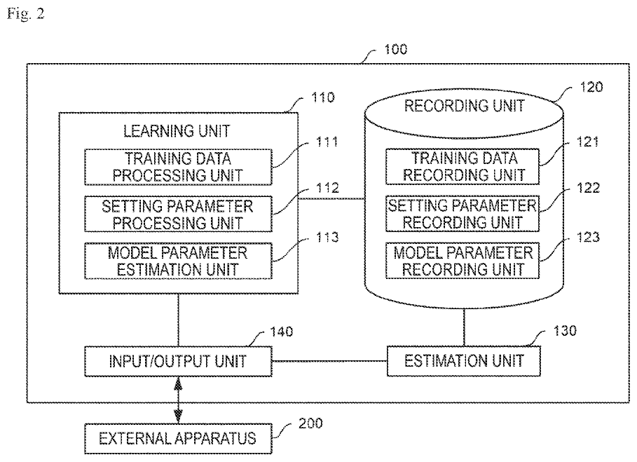

FIG. 2 is a block diagram illustrating a functional component of the estimation apparatus 100. As illustrated in FIG. 2, the estimation apparatus 100 includes a learning unit 110, a recording unit 120, an estimation unit 130, and an input/output unit 140.

The learning unit 110 learns a relationship between a tensor representing an activity log of a user and a tensor representing an acceptability log of the user. In the present embodiment, the tensor representing the activity log is also referred to as an activity tensor, and the tensor representing the acceptability log is also referred to as an acceptability tensor. The activity tensor may be a tensor including information about the number of times the user switches activities in a predetermined time frame as described below. The activity tensor may also be a tensor including information about a duration of an activity as described below. The activity tensor may also be a tensor including information about the number of times an activity is performed as described below. The acceptability tensor may be a tensor including information representing whether it is acceptable for a user to switch activities in a predetermined time frame. The acceptability tensor may also be a tensor including information about an allowable duration of an activity. The acceptability tensor may also be a tensor including information about the allowable number of times the activity is performed. The learning unit 110 includes a training data processing unit 111, a setting parameter processing unit 112, and a model parameter estimation unit 113. The recording unit 120 records various data and parameters. The recording unit 120 includes a training data recording unit 121, a setting parameter recording unit 122, and a model parameter recording unit 123.

The training data processing unit 111 stores training data including a set of an activity tensor and an acceptability tensor in the training data recording unit 121. The setting parameter processing unit 112 stores a setting parameter in the setting parameter recording unit 122. Here, the setting parameter is a parameter used for estimating the acceptability tensor described below. Examples of the setting parameter will be described below.

The model parameter estimation unit 113 estimates a model parameter by receiving the training data and the setting parameter recorded in the recording unit 120. The model parameter estimation unit 113 stores the estimated model parameter in the model parameter recording unit 123. The model parameter is a parameter used for estimating an acceptability tensor when an activity log without a corresponding acceptability log is input to the estimation apparatus 100. Examples of the model parameter will be described below.

The estimation unit 130 estimates an acceptability tensor from an activity tensor representing an activity log without a corresponding acceptability log. The estimation unit 130 uses the model parameter stored in the model parameter recording unit 123 to estimate the acceptability tensor. A process for estimating the acceptability tensor will be described below.

The input/output unit 140 receives training data and the activity tensor representing the activity log without a corresponding acceptability log. Subsequently, the input/output unit 140 outputs the acceptability tensor estimated by the estimation unit 130 from the activity log without a corresponding acceptability log. The input/output unit 140 receives and outputs data or the like from and to the external apparatus 200 different from the estimation apparatus 100.

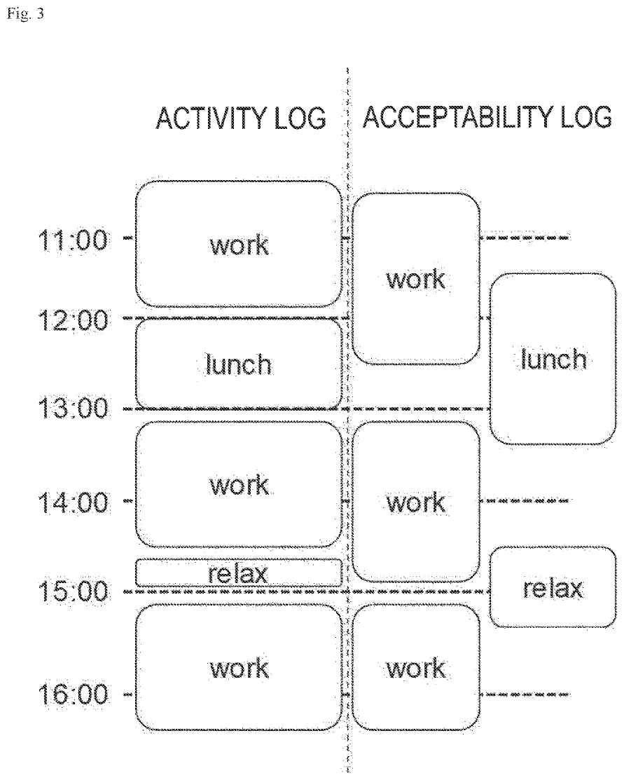

FIG. 3 is a diagram illustrating an example of an activity log and an acceptability log. The activity log and the acceptability log will be described with reference to FIG. 3.

In the example of FIG. 3, a log including “work” until 12:00, “lunch” from 12:00 to 13:00, “work” from 13:00 to 14:45, “relax” from 14:45 to 15:00, and “work” from 15:00 is recorded in an activity log of a user. In NPL 1 mentioned above, the activity log is data created on the basis of a self-assessment of a user. In the present embodiment, the activity log may similarly be data created on the basis of a self-assessment of a user, or may be data automatically created by utilizing an activity recognition technique utilizing a sensor such as a sensor of a smartphone or a smart watch.

Furthermore, in the example of FIG. 3, the acceptability log of the user records a log that it was acceptable to extend the work in the morning until 12:30, and to shift the time for lunch to any time in a range from 11:30 to 13:30. In the present embodiment, the acceptability log is data created on the basis of a self-assessment of a user.

As described in Reference Literature 1 and 2, the activity log and the acceptability log may be represented as tensors.
[Reference Literature 1] Masahiro Kohjima, Tatsushi Matsubayashi, Hiroshi Sawada. Multiple Data Analysis and Non-negative Matrix/Tensor Factorization [I]: Multiple Data Analysis and Its Advances, The Journal of the Institute of Electronics, Information and Communication Engineers, 99(6): 543-550, 2016.
[Reference Literature 2] Tatsushi Matsubayashi, Masahiro Kohjima, Hiroshi Sawada. Multiple Data Analysis and Non-negative Matrix/Tensor Factorization [II⋅Finish]: Tensor Data Analysis and Applications, The Journal of the Institute of Electronics, Information and Communication Engineers, 99(7): 691-698, 2016.

Note that, in the estimation apparatus 100 according to the present embodiment, the activity log and the acceptability log can be represented as tensors. There are various ways to represent the logs as tensors. Depending on what kind of information is represented and what parameter an axis represents, the activity log and the acceptability log can be represented as various tensors.

For example, when the activity log on a certain day is represented as an activity tensor, the activity log may be represented as a second rank tensor (matrix) in which a row is a time frame, a column is an activity, and an element is 1 if the activity is performed in the time frame and 0 if the activity is not performed in the time frame. When the activity log is represented as a second rank tensor, the number of times a user switches activities in a certain time frame is seen by comparing the elements in the activity tensor. Consequently, the activity tensor may be a tensor including information about the number of times the user switches activities in a certain time frame.

Furthermore, when the activity log on a certain day is represented as an activity tensor, the activity log may also be represented as a second rank tensor (matrix) in which a row represents a time frame, a column represents an activity, and an element represents a duration during which the activity is performed in the time frame, for example. For example, when a maximum value (60 minutes) of the duration, which is an element of the tensor, is 1 and the duration is 45 minutes, the duration may be set to 45 (minutes)/60 (minutes)=0.75.

Furthermore, when the activity log on a certain day is represented as a tensor, the activity log may also be represented as a third rank tensor in which a row is a time frame, a column is a previous activity, a depth is a later activity, and an element is 1 if a user switches activities from the previous activity to the later activity in the time frame and 0 if the user does not switch activities. When the activity log is represented as a third rank tensor, the number of times the user switches activities in a certain time frame is obtained by summing the element in the activity tensor. Consequently, the activity tensor may be a tensor including information about the number of times the user switches activities in a certain time frame.

Furthermore, when the activity log on a certain day is represented as a tensor, the activity log may also be represented as a third rank tensor in which a row represents a time frame, a column represents a previous activity, a depth represents a later activity, and an element represents the number of times a user switches activities from the previous activity to the later activity in the time frame.

Furthermore, when the activity log on a certain day is represented as a tensor, the activity log may also be represented as a fourth rank tensor in which a first axis represents a user, a second axis represents a time frame, a third axis represents a previous activity, a fourth axis represents a later activity, and an element represents the number of times a user switches activities from the previous activity to the later activity in the time frame.

Substantially similarly to the activity log described above, the acceptability log may be represented as a second rank tensor (matrix) in which a row is a time frame, a column is an activity, and an element is 1 if it is acceptable for the user to perform the activity in the time frame and 0 if it is not acceptable for the user to perform the activity in the time frame. Furthermore, the acceptability log may also be represented as a third rank tensor in which a row is a time frame, a column is a previous activity, a depth is a later activity, and an element is 1 if it is acceptable for the user to switch activities from the previous activity to the later activity in the time frame and 0 if it is not acceptable for the user to switch activities. That is, the acceptability tensor may be a tensor including information representing whether it is acceptable for a user to switch activities in a certain time frame. Furthermore, when the acceptability log on a certain day is represented as a tensor, the acceptability log may also be represented as a second rank tensor (matrix) in which a row represents a time frame, a column represents an activity, and an element represents a duration during which it is acceptable for the user to perform the activity in the time frame. For example, when a maximum value (60 minutes) of the duration during which it is acceptable for the user to perform an activity and which is an element of the tensor, is 1 and it is acceptable for the user to perform the activity for 45 minutes, the duration may be set to 45 (minutes)/60 (minutes)=0.75. Furthermore, when the acceptability log on a certain day is represented as a tensor, the acceptability log may also be represented as a third rank tensor in which a row represents a time frame, a column represents a previous activity, a depth represents a later activity, and an element represents the number of times it is acceptable for the user to switch activities from the previous activity to the later activity in the time frame.

In any of the above representations, the activity log can be represented as an activity tensor and the acceptability log can be represented as an acceptability tensor. Even when the activity log and the acceptability log are obtained for each of a plurality of users, the activity log and the acceptability log can be represented as a tensor in which the number of axes for the users is increased. In the following description, the activity log and the acceptability log will be mainly represented as the third rank activity tensor and acceptability tensor mentioned above. However, even when another tensor is used to represent the activity log and the acceptability log, it is possible to use an approach in much the same way as the following technique.

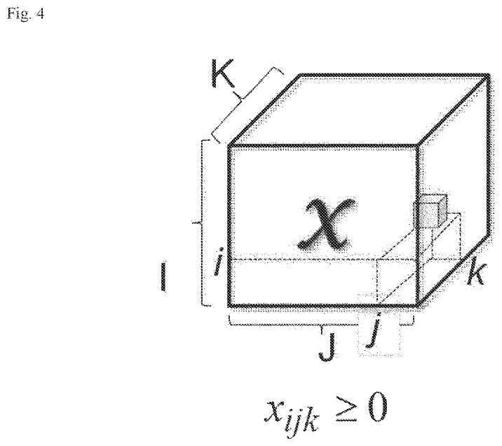

In the present embodiment, the activity tensor representing the activity log is represented by X={xᵢⱼₖ}, and the acceptability tensor representing the acceptability log is represented by Y={yᵢⱼₖ}. FIG. 4 is a diagram illustrating a state where an activity log is represented as a tensor. In the present embodiment, it is assumed that training data D, which is a set of m=1, . . . , n activity logs and acceptability logs, is given. The training data D is defined by the following equation.

[symbol]

={X^{m},Yₘ}ₘ₌₁ⁿ [Math. 1]

X^{m} and Y^{m} in the above equation are defined respectively by the following equations.

X^{m}={xᵢⱼₖ^{m}},Y^{m}={yᵢⱼₖ^{m}} [Math. 2]

As described above, an example is assumed in which x^{m}ᵢⱼₖ is a tensor representing, in m-th activity log data, the number of times a user switches activities from an activity j to an activity k in a time frame i. Furthermore, an example is assumed in which y^{m}ᵢⱼₖ is a tensor including, in m-th acceptability log data, a value of 1 if the user can accept whether the user switches activities from the activity j to the activity k in the time frame i, and a value of 0 if the user cannot accept whether the user switches activities.

Note that m-th data may be data corresponding to a certain day, or may be data calculated from a summary value of data collected throughout one week. In the latter case, x^{m}ᵢⱼₖ may be a proportion at which the activity k is an activity performed after the activity j in the time frame i in the activity log of the one week, for example.

In the present embodiment, the estimation apparatus 100 learns a relationship between an activity tensor X representing an activity log and an acceptability tensor Y representing an acceptability log. Subsequently, the estimation apparatus 100 uses a learning result to estimate an acceptability tensor for an activity tensor representing an activity log without a corresponding acceptability log. The estimation apparatus 100 learns the relationship between the activity tensor X and the acceptability tensor Y by deep learning or tensor factorization. The deep learning is generally an effective method when a large amount of the training data D can be utilized. The tensor factorization is a method considered to be effective when the data amount of the training data D is small as compared with the deep learning. Whether the deep learning or the tensor factorization is to be used also depends on the size of the tensor to be input to the estimation apparatus 100 or to be output from the estimation apparatus 100. When the time frames are very narrowly divided and the activities are very narrowly divided, the size of the tensor is large, and thus, a larger amount of the training data D is required than in a case where the size of the tensor is small. The estimation apparatus 100 may select either the deep learning or the tensor factorization, by attempting both the deep learning and the tensor factorization so as to utilize the method capable of estimating the acceptability log best by cross-validation or the like.

(1) Deep Learning

First, a case where deep learning is used to estimate the acceptability log is described. A deep learning model including a parameter θ is represented as f_{θ}: X->Y. θ represents all parameters estimated from the data, such as a network weight parameter and a parameter used in a convolution calculation. Consequently, when deep learning is used, the model parameter estimation unit 113 uses the training data D to estimate the parameter θ. Furthermore, when deep learning is used, a network structure of deep learning corresponds to a setting parameter. The model parameter estimation unit 113 estimates the parameter θ by minimizing a loss function of the following equation, for example.

[symbol]

(θ)=Σₘ₌₁ⁿ∥y^{m}−ƒ_{θ}(X^{m})∥₂² [Math. 3]

In the above equation, the following expression represents the square of an L₂ norm.

∥⋅∥₂² [Math. 4]

The square of the L₂ norm of the acceptability tensor Y is obtained according to the following equation.

∥Y∥₂²=Σ_{i,j,k}yᵢⱼₖ² [Math. 5]

In the present embodiment, a case where a squared error is utilized as an objective function is described, but the present disclosure is not limited to this example. The estimation apparatus 100 may utilize any error function, such as an absolute error, instead of the squared error.

When the model parameter estimation unit 113 learns the relationship between the activity tensor X and the acceptability tensor Y by deep learning, the parameter θ derived from the learning is recorded in the model parameter recording unit 123. The estimation unit 130 can use the parameter θ recorded in the model parameter recording unit 123 to estimate an acceptability tensor for an activity tensor representing an activity log without a corresponding acceptability log.

(2) Tensor Factorization

Next, a case where tensor factorization is used to estimate the acceptability log is described. The tensor factorization refers to a technique in which a tensor to be input is factorized into a plurality of (low order) matrices or tensor products (Reference Literature 1 and 2). Hereinafter, an activity tensor representing an activity log is represented by X={xᵢⱼₖ}, and an acceptability tensor representing an acceptability log is represented by Y={yᵢⱼₖ}. The tensor factorization can be applied if at least one set of the activity tensor X and the acceptability tensor Y is given. For the sake of simplicity, a situation where one set of training data D={X, Y} is given will be described. However, needless to say, the tensor factorization may also be applied to a situation where a plurality of sets of data are given.

Pattern Extraction by Tensor Factorization

First, a pattern extraction by a general tensor factorization will be described. The tensor factorization includes various factorization methods such as canonical polyadic (PC) decomposition and Tucker decomposition. Any factorization system may be utilized in the present embodiment. Hereinafter, description proceeds by referring to an example in which the activity tensor X is factorized into a (mode) product of a tensor B={bⱼₖᵣ}_{j, k, r} and a matrix A={aᵢᵣ}_{i, r}. An approximation value of an element of the activity tensor X to be input, including the factorization result, is represented by the following equation.

\hat{X}={\hat{x}ᵢⱼₖ}_{i,j,k} [Math. 6]

Hereinafter, in the expressions, a character added with “\hat{ }” above the symbol (for example, X) is sometimes represented as \hat{ }X in the following. An element of \hat{ }X is given by Equation (1) below.

[Math. 7]

\hat{x}ᵢⱼₖ=Σᵣ₌₁^{R}aᵢᵣbⱼₖᵣ (1)

The matrix A and the tensor B are estimated so that the element xᵢⱼₖ of the activity tensor X to be input and \hat{ }xᵢⱼₖ being an approximation value of the element xᵢⱼₖ are close values.

The squared error, the generalized KL divergence, the Itakura-Saito divergence, and the like are employed for a scale to indicate how close the values are. In a case where the generalized KL divergence is employed, the scale is defined by the following equation with xᵢⱼₖ and \hat{ }xᵢⱼₖ.

$${d}_{KL} ( {x}_{ijk} \vert \vert {\hat{x}}_{ijk} ) = {x}_{ijk} log \frac{{x}_{ijk}}{{\hat{x}}_{ijk}} - {x}_{ijk} + {\hat{x}}_{ijk} \qquad [ Math . 8 ]$$

When the generalized KL divergence is used as a scale, the sum of all the elements is used as a loss function, as indicated in the following equation.

D_{KL}(X∥\hat{X})=Σ_{i,j,k}d_{KL}(xᵢⱼₖ∥\hat{x}ᵢⱼₖ) [Math. 9]

When this loss function is used to solve the following optimization problem, the matrix A and the tensor B can be obtained from the activity tensor X.

minimize D_{KL}(X∥X)s.t. A≥0,B≥0 [Math. 10]

Note that A≥0 and B≥0 represent non-negative constraints indicating that the elements of the matrix and the tensor are each 0 or more. The tensor factorization may be performed without imposing the non-negative constraints. However, by the constraints, an element aᵢᵣ of the matrix A can be regarded as representing a degree of the probability at which an activity belongs to a cluster r of the time frame i, and an element bⱼₖᵣ of the tensor B can be regarded as representing a degree of the frequency at which the activity j switches to the activity k in the cluster r. Consequently, in the present embodiment, tensor factorization with the non-negative constraints is employed. If non-negative tensor factorization is applied to a tensor representing an activity log, elements can be divided into a cluster corresponding to a time frame (7:00 and 8:00) during winch a user prepares to go to work and a cluster corresponding to a time frame during work, for example. In this case, it can be expected that transitions between characteristic activities of each cluster, such as a transition from having breakfast to going to work and a transition from working to taking a break, are extracted.

(2-1) Learning Hyperparameter of Graph Laplacian Regularization

A technique is described in which the tensor factorization approach mentioned above is utilized and the activity tensor X is input to output the acceptability tensor Y corresponding to the activity tensor X. First, a technique using graph Laplacian regularization will be described.

In tensor factorization and matrix factorization, graph Laplacian regularization is utilized with the object of reflecting, in the factorization result, prior knowledge that an i-th row (aᵢₗ, . . . , a_{iR}) and an i′-th row (a_{i′1}, . . . , a_{i′R}) of a matrix (for example, the matrix A) being the factorization result should be similar (see Reference Literature 3). Specifically, in graph Laplacian regularization, the object mentioned above is achieved by adding, to the objective function, a regularization term for a degree of similarity between values that should be similar.
[Reference Literature 3] Deng Cai, Xiaofei He, Jiawei Han, and Thomas S Huang. Graph regularized nonnegative matrix factorization for data representation. IEEE transactions on pattern analysis and machine intelligence, 33(8): 1548-1560, 2010.

A regularization term is defined by using a matrix W indicating a relationship of elements that should be similar. The matrix W is defined as described below. The matrix W is an example of parameters recorded by the setting parameter processing unit 112 in the setting parameter recording unit 122.

W∈ [symbol] ^{I×I} [Math. 11]

The matrix W may be created by various methods. These methods include a 0-1 weighting in which wᵢₗ=1 if i and 1 should be similar and wᵢₗ=0 if i and l should not be similar, heat kernel weighting, and dot-product weighting (see Reference Literature 3). The regularization term is defined by the following equation.

$$\begin{matrix}Ω ( A ; W ) = \frac{1}{2} \sum \text{?} ( {𝒟}_{KL} ( \text{?} ) + {𝒟}_{KL} ( \text{?} ) ) \text{?} \\ = \frac{1}{2} \sum \text{?} {\sum }^{R} \text{?} ( \text{?} log \text{?} + \text{?} log \text{?} ) \text{?}\end{matrix} \qquad [ Math . 12 ]$$

$$\text{?} \text{indicates text missing or illegible when filed}$$

The meaning of this regularization term can be easily understood by imagining 0-1 weighting. If i and l do not need to be similar, wᵢₗ=0, and thus, the divergence of aᵢ and aₗ does not contribute to the value. However, if wᵢₗ≠0, the objective function is smaller as the divergence of aᵢ and aₗ is smaller.

The estimation apparatus 100 according to the present embodiment uses graph Laplacian regularization to output the acceptability tensor Y from the activity tensor X. In the relationship between the activity log and the acceptability log illustrated in FIG. 3, as represented by the log “lunch”, an activity start time and an activity end time of the acceptability log encompass an activity start time and an activity end time of the activity log. Consequently, when the activity tensor X is subjected to tensor factorization, the constraint that the values of the matrix A in earlier and later time frames are similar is imposed. It is assumed that, by this constraint, it is possible to estimate the matrix A and the tensor B so that \hat{ }x corresponds to a tensor representing the acceptability log.

This is because, if {aᵢᵣ}^{R}ᵣ₌₁ and {a_{i-1, r}}^{R}ᵣ₌₁ are similar, {xᵢⱼₖ}_{j, k} and {x_{i-1, jk}}_{j,k} are also similar, and if a transition between activities from “work” to “lunch” occurs between 12:00 and 13:00, an estimation result that an activity from “work” to “lunch” also occurs between 11:00 and 12:00 is obtained. Note that, in order to obtain this estimation, it is only required to set the matrix W so that w_{ii′} is larger as the time frame i and a time frame i′ are closer to each other, and is closer to 0 as the time frame i and the time frame i′ are farther from each other. For example, it is conceivable to set the matrix W so that only 1 elements before and after the time frame i are 1, that is, w_{ii′}=1 if the expression i′∈{i−1, i−1+1, . . . , i, . . . , i+1−1, i+1} is satisfied, and w_{ii′}=0, otherwise. Note that, if i−1<1, the expression i′∈{i−1+I, i−1+1+I, . . . , 1, . . . , i, . . . , i+1−1, i+1} may be employed in which I is added to an element smaller than 1, so that i′ follows the matrix indexes in reverse. Similarly, when i+1>I, the expression i′∈{i−1+I, i−1+1+I, . . . , i, . . . , I, . . . , i+1−1−I, i+1−I} may be employed in which I is subtracted from elements larger than I.

In the tensor factorization using graph Laplacian regularization, the objective function indicated in Expression (2) below is minimized.

[Math. 13]

minimize_{A,B}{D_{DL}(X∥\hat{X})+αΩ(A;W)}s.t. A≥0,B≥0 (2)

α is a hyperparameter determining an extent to which the regularization term contributes to the objective function, and is an example of a parameter recorded by the setting parameter processing unit 112 in the setting parameter recording unit 122. Any optimization procedure such as a gradient method, Newton's method, and an auxiliary function method may be employed for optimization. The acceptability tensor Y corresponding to the activity tensor X used as an input is utilized to determine this hyperparameter. Specifically, the hyperparameter is determined by selecting, from Q candidate values {α_{Q}}^{Q}_{q=1}, a candidate value in which a tensor created from matrices obtained by solving the above-described optimization problem using each of the candidate values, is most similar to the acceptability log. The tensor created from the matrices obtained by solving the optimization problem is represented as follows.

\hat{X}^{(α}^{_{q}}⁾ [Math. 14]

The hyperparameter α is determined by Equation (3) below.

[Math. 15]

α=arg min_{q=1, . . . ,Q}D_{KL}(Y∥\hat{X}^{(α}^{_{q}}⁾) (3)

In this Equation (3), the most suitable hyperparameter is selected in the conversion from the activity tensor to the acceptability tensor. Thus, when this hyperparameter is obtained, even if an activity log Xₜₑₛₜ without a corresponding acceptability log is given, it is possible to obtain the acceptability tensor by reusing this hyperparameter to perform tensor factorization using graph Laplacian regularization. Specifically, the model parameter estimation unit 113 reuses the hyperparameter α obtained by the above-described method and an estimation result B of the optimization problem indicated by Expression (2) to solve Expression (4) below. The model parameter estimation unit 113 may simultaneously estimate the tensor B.

[Math. 16]

minimize_{A}{D_{KL}(Xₜₑₛₜ∥\hat{X})+αΩ(A;W)}s.t. A≥0 (4)

The model parameter estimation unit 113 may use a tensor calculated by using the matrix A (referred to as Aₜₑₛₜ) obtained by solving Expression (4) and the existing tensor B, as an estimation value of the acceptability tensor having the activity log Xₜₑₛₜ as an input. The matrix Aₜₑₛₜ is defined by the following equation.

Aₜₑₛₜ={aᵢᵣ^{test}}[Math. 17]

The estimation value of the acceptability tensor having the activity log Xₜₑₛₜ as an input is determined by Equations (5) below.

[Math. 18]

\hat{X}ₜₑₛₜ={\hat{x}ᵢⱼₖ^{test}}ᵢⱼₖ

\hat{x}ᵢⱼₖ^{test}=Σᵣ₌₁^{R}aᵢᵣ^{test}bⱼₖᵣ (5)

The estimation unit 130 can estimate the acceptability log from an activity log without a corresponding acceptability log, by using the estimation value of the acceptability tensor determined by Equations (5). Note that, even when a factorized form different from Equation (1) is employed for a factorized form during tensor factorization, the model parameter estimation unit 113 can obtain the estimation value of the acceptability tensor by substantially the same method.

(2-2) Learning Correspondence Relationship of Factor Matrices

The method described below is a method of learning, from training data, a matrix C explicitly representing a correspondence relationship for each row between approximation values of elements of an input tensor and approximation values of elements of an output tensor. In the present embodiment, a correspondence relationship between I earlier and later time frames i is considered. The matrix C is a matrix in which an element c_{ii′} is 0 when the following condition is satisfied.

c_{ii′}=0 if (i′∉{i− [symbol] ,i− [symbol] +1, . . . ,i, . . . ,i+ [symbol] −1,i+ [symbol] }) [Math. 19]

The matrix C is defined as a matrix having only I(2l+1) elements other than the elements mentioned above, as parameters. Here, l which determines an element being a non-zero component of the matrix C, is an example of a parameter recorded by the setting parameter processing unit 112 in the setting parameter recording unit 122. However, when i−l<1, for example, a set {i−l+I, i−l+1+I, . . . , 1, . . . , i, . . . , i+l−1, i+l} may be employed in which I is added to elements smaller than 1, so that i′ follows the matrix indexes in reverse. Similarly, when i+l>I, a set {i−l+1, i−l+1+I, . . . , i, . . . , I, . . . , i+l−1−I, i+l−I} may be employed in which i is subtracted from elements larger than I.

Hereinafter, for the sake of simplicity, the entire space that may be taken by the matrix C in which the constraint that a part of the elements mentioned above is always 0 is satisfied, and in which the other elements are always 0 or more, is represented below.

[symbol]

[Math. 20]

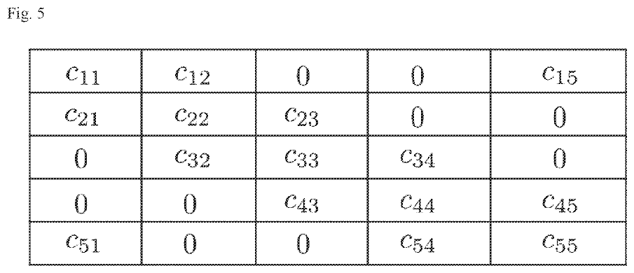

FIG. 5 is a specific example of the matrix C when I=5 and l=1. A simultaneous factorization system in which approximation values of the tensor to be input are constructed as described below is considered.

\hat{X}={\hat{x}ᵢⱼₖ}ᵢⱼₖ,ŷ={yᵢⱼₖ}ᵢⱼₖ

\hat{x}ᵢⱼₖ=Σᵣ₌₁^{R}aᵢᵣbⱼₖᵣ,ŷᵢⱼₖ=Σᵣ₌₁^{R}Σ_{i′=1}^{I}c_{ii′}a_{i′r}bⱼₖᵣ [Math. 21]

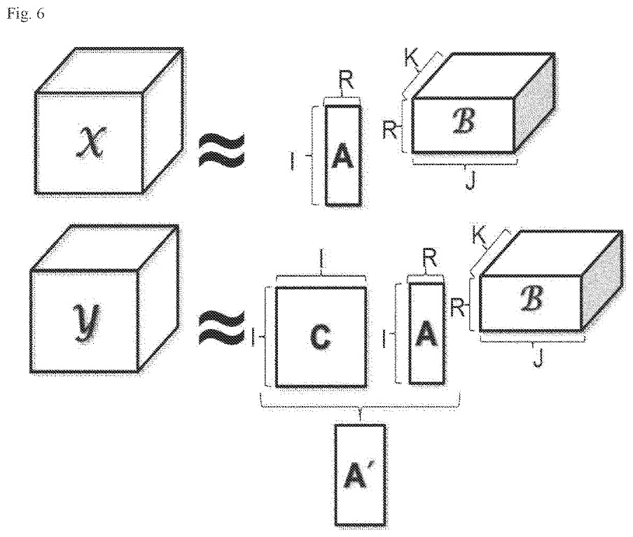

aᵢᵣ and bⱼₖᵣ used for the factorization of each tensor are jointly used. FIG. 6 is a diagram illustrating a factorization system. In the matrix A, the number of rows is I and the number of columns is R. The tensor B is a third rank tensor in which the number of rows is J, the number of columns is K. and the number of depths is R. For the sake of convenience, if the product of the matrices C and A is written as A′(=CA), a′ᵢᵣ is represented as follows, from the definition of the matrix C.

aᵢᵣ′=Σ_{i′=1}^{I}c_{ii′}a_{i′r}=Σi′∈ [symbol] c_{ii′}a_{i′r} [Math. 22]

That is, the (i, r)-th component of the matrix A′ is represented as a weighted sum of the (i−1, r), . . . , (i, r), . . . , (i+1, r)-th components of the matrix A. The model parameter estimation unit 113 estimates the matrices A and C, and the tensor B by solving the optimization problem represented by Expression (6) below.

[Math. 23]

minimize_{A,B,C}{D_{KL}(X∥\hat{X})+D_{KL}(Y∥Ŷ)}s.t. A≥0,B≥0,C∈ [symbol] (6)

Any optimization procedure such as a gradient method, Newton's method, and an auxiliary function method may be employed for the optimization. Consequently, when the matrices A and C, and the tensor B are obtained, even if the activity log Xₜₑₛₜ without a corresponding acceptability log is given, it is possible to reuse this value to obtain the acceptability tensor. Specifically, the model parameter estimation unit 113 reuses estimation results B and C of the optimization problem in Expression (6) to firstly solve Expression (7) below. The model parameter estimation unit 113 may simultaneously estimate the tensor B.

[Math. 24]

minimize_{A}{D_{DL}(Xₜₑₛₜ∥\hat{x})}s.t. A≥0 (7)

The model parameter estimation unit 113 can use a solution Aₜₑₛₜ of Expression (7) to determine an estimation value of the acceptability tensor according to the following equations. The model parameter estimation unit 113 determines, from Equations (8) below, an estimation value of an acceptability tensor having the activity log Xₜₑₛₜ as an input.

[Math. 25]

Ŷₜₑₛₜ={ŷᵢⱼₖ^{test}}

ŷᵢⱼₖ=Σᵣ₌₁^{R}Σ_{i′=1}^{I}cᵢᵢ,α_{i′r}^{test}bⱼₖᵣ (8)

When the estimation value of the acceptability tensor having the activity log Xₜₑₛₜ as an input is determined, the estimation unit 130 can calculate an acceptability tensor from the activity tensor Xₜₑₛₜ representing an activity log without a corresponding acceptability log.

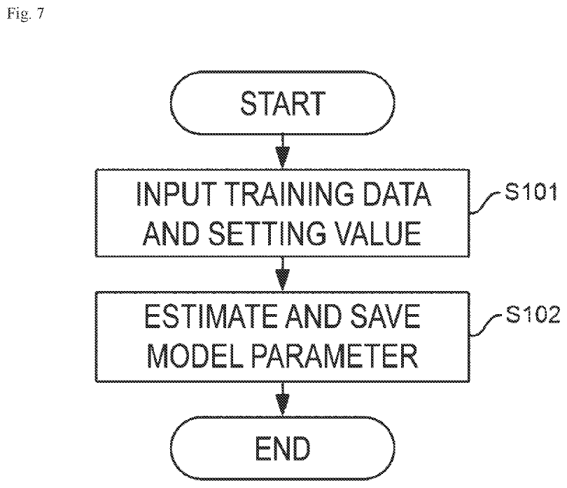

Subsequently, an operation of the estimation apparatus 100 according to the present embodiment will be described. FIG. 7 is a flowchart illustrating a model parameter learning process of the estimation apparatus 100 according to the present embodiment. The model parameter learning process is realized by the CPU 11 reading the estimation program stored in the ROM 12 or the storage 14 and loading and executing the estimation program into and in the RAM 13.

The CPU 11 receives the training data D and the setting parameter as an input, and stores the received training data D and setting parameter in the storage 14 (step S101). As described above, the training data D is data including the activity tensor X and the acceptability tensor Y. When deep learning is used, the setting parameter is a network structure of deep learning, and when graph Laplacian regularization is used, the setting parameter is the matrix W representing the relationship between elements that should be similar. Furthermore, when a correspondence relationship between factor matrices is used, the setting parameters are variables determining elements being non-zero components of the matrix C representing a correspondence relationship for each row between approximation values of elements of an input tensor and approximation values of elements of an output tensor.

Following step S101, the CPU 11 estimates model parameters by using the training data D and the setting parameter, and stores the estimated model parameters in the storage 14 (step S102). The model parameters are estimated by deep learning or tensor factorization, as described above.

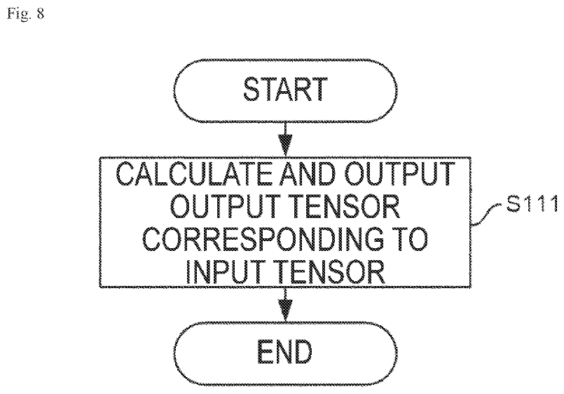

FIG. 8 is a flowchart illustrating a test data process of the estimation apparatus 100 according to the present embodiment. The test data process is realized by the CPU 11 reading the estimation program stored in the ROM 12 or the storage 14 and loading and executing the estimation program into and in the RAM 13.

The CPU 11 uses the model parameter stored in the storage 14 to calculate an output tensor (an estimation value of the acceptability tensor representing the acceptability log) corresponding to the input tensor (the activity tensor representing the activity log) (step S111). Here, the input tensor is the activity tensor Xₜₑₛₜ representing an activity log without a corresponding acceptability log.

When deep learning is used, the CPU 11 calculates f_{θ}(Xₜₑₛₜ) to obtain the output tensor. Furthermore, when the hyperparameter of graph Laplacian regularization is used, the CPU 11 obtains the matrix Aₜₑₛₜ and the tensor B from the activity tensor Xₜₑₛₜ by solving the optimization problem to calculate the estimation value of the acceptability tensor of Equations (5) mentioned above. Moreover, when the correspondence relationship of the factor matrices is used, the CPU 11 obtains the matrix Aₜₑₛₜ and the tensor B from the activity tensor Xₜₑₛₜ by solving the optimization problem to calculate the estimation value of the acceptability tensor representing the acceptability log of Equations (8) mentioned above.

The estimation apparatus 100 according to the present embodiment can estimate the acceptability tensor from an activity log without a corresponding acceptability log, to finally estimate the acceptability log. It is possible to estimate the acceptability log from the activity log, and thus, in a case of intervening in a user to encourage the user to perform a certain activity during a certain time frame on the day (for example, when a notification is sent via a smartphone, or when a person actually says something to the user), it can be seen whether the user accepts the intervention. Subsequently, the estimation apparatus 100 according to the present embodiment estimates an acceptability log from an activity log without a corresponding acceptability log, and thus, in order to improve the daily life of the user, the acceptability log can be utilized to determine how another person should intervene to encourage the user.

Any technique, such as a gradient method, may be employed for the optimization in the embodiments described above. Furthermore, any network structure may be employed in deep learning and any factorized form may be employed in the tensor factorization. Similarly, any regularization term can be employed for the regularization term of the objective function.

Note that, in each of the above-described embodiments, various types of processors other than the CPU may execute the learning process and the estimation process which the CPU executes by reading software (a program). Examples of the processor in such a case include a programmable logic device (PLD) such as a field-programmable gate array (FPGA) the circuit configuration of which can be changed after manufacturing, a dedicated electric circuit such as an application specific integrated circuit (ASIC) that is a processor having a circuit configuration designed dedicatedly for executing the specific processing, and the like. The learning process and the estimation process may be executed by one of these various types of processors or may be executed by a combination of two or more processors of the same type or different types (for example, a plurality of FPGAs and a combination of a CPU and an FPGA). More specifically, the hardware structure of such various processors is an electrical circuit obtained by combining circuit devices such as semiconductor devices.

In the embodiment described above, an aspect has been described in which the estimation program is stored (installed) in advance in the storage 14, but the embodiment is not limited to this aspect. The program may be provided in the form of being stored in a non-transitory storage medium such as a compact disk read only memory (CD-ROM), a digital versatile disk read only memory (DVD-RAM), or a universal serial bus (USB) memory. The program may be in a form that is downloaded from an external apparatus via a network.

With respect to the above embodiment, the following supplements are further disclosed.

### Supplementary Note 1

An estimation apparatus including
a memory and
at least one processor connected to the memory,
in which the at least one processor is configured to estimate an acceptability tensor for to an activity tensor representing an activity log without a corresponding acceptability log, by using a learning result of a relationship between an activity tensor representing an activity log recording activities of a user, and an acceptability tensor representing an acceptability log recording an acceptability for a time change of the activities of the user.

### Supplementary Note 2

A non-temporary storage medium storing a program executable by a computer to execute an estimation process,
in which the estimation process estimates an acceptability tensor for an activity tensor representing an activity log without a corresponding acceptability log, by using a learning result of a relationship between an activity tensor representing an activity log recording activities of a user, and an acceptability tensor representing an acceptability log recording an acceptability for a time change of the activities of the user.

### REFERENCE SIGNS LIST

100 Estimation apparatus
110 Learning unit
111 Training data processing unit
112 Setting parameter processing unit
113 Model parameter estimation unit
120 Recording unit
121 Training data recording unit
122 Setting parameter recording unit
123 Model parameter recording unit
130 Estimation unit
140 Input/output unit
200 External apparatus

1. An estimation apparatus, comprising:
an estimation unit configured to estimate an acceptability tensor corresponding to an activity tensor representing an activity log without a corresponding acceptability log, by using a result of previously performed learning of a relationship between an activity tensor representing an activity log recording activities of a user, and an acceptability tensor representing an acceptability log recording an acceptability for a time change of the activities of the user.

2. The estimation apparatus according to claim 1, wherein
the activity tensor includes information about the number of times the user switches activities in a predetermined time frame, and
the acceptability tensor includes information representing whether it is acceptable for the user to switch activities in the predetermined time frame.

3. The estimation apparatus according to claim 2, further comprising a learning unit configured to learn a relationship between the activity tensor and the acceptability tensor by tensor factorization of the activity tensor.

4. The estimation apparatus according to claim 3, wherein the learning unit learns a relationship between the activity tensor and the acceptability tensor by tensor factorization of the activity tensor using graph Laplacian regularization.

5. The estimation apparatus according to claim 3, wherein the learning unit learns a relationship between the activity tensor and the acceptability tensor by tensor factorization of the activity tensor using a matrix representing a correspondence relationship between an approximation value of the activity tensor and an approximation value of the acceptability tensor.

6. The estimation apparatus according to claim 1, further comprising:
a learning unit configured to learn a relationship between the activity tensor and the acceptability tensor by using deep learning.

7. An estimation method comprising, at a computer, including:
estimating an acceptability tensor corresponding to an activity tensor representing an activity log without a corresponding acceptability log, by using a result of previously performed learning of a relationship between an activity tensor representing an activity log recording activities of a user, and an acceptability tensor representing an acceptability log recording an acceptability for a time change of the activities of the user.

8. A non-transitory computer-readable medium having computer-readable instructions stored thereon, which, when executed, cause a computer including a memory and a processor to execute a set of operations, comprising:
estimating an acceptability tensor corresponding to an activity tensor representing an activity log without a corresponding acceptability log, by using a result of previously performed learning of a relationship between an activity tensor representing an activity log recording activities of a user, and an acceptability tensor representing an acceptability log recording an acceptability for a time change of the activities of the user.

9. The estimation method according to claim 7, wherein
the activity tensor includes information about the number of times the user switches activities in a predetermined time frame, and
the acceptability tensor includes information representing whether it is acceptable for the user to switch activities in the predetermined time frame.

10. The estimation method according to claim 9, further comprising:
the learning of the relationship between the activity tensor and the acceptability tensor learns by tensor factorization of the activity tensor.

11. The estimation method according to claim 10, wherein:
the learning of the relationship between the activity tensor and the acceptability tensor by tensor factorization of the activity tensor uses graph Laplacian regularization.

12. The estimation method according to claim 10, wherein:
the learning of the relationship between the activity tensor and the acceptability tensor by tensor factorization of the activity tensor uses a matrix representing a correspondence relationship between an approximation value of the activity tensor and an approximation value of the acceptability tensor.

13. The estimation method according to claim 7, further comprising:
learning the relationship between the activity tensor and the acceptability tensor by using deep learning.

14. The non-transitory computer-readable medium according to claim 8, wherein
the activity tensor includes information about the number of times the user switches activities in a predetermined time frame, and
the acceptability tensor includes information representing whether it is acceptable for the user to switch activities in the predetermined time frame.

15. The non-transitory computer-readable medium according to claim 14, wherein the learning of the relationship between the activity tensor and the acceptability tensor learns by tensor factorization of the activity tensor.

16. The non-transitory computer-readable medium according to claim 15, wherein:
the learning of the relationship between the activity tensor and the acceptability tensor by tensor factorization of the activity tensor uses graph Laplacian regularization.

17. The non-transitory computer-readable medium according to claim 15, wherein:
the learning of the relationship between the activity tensor and the acceptability tensor by tensor factorization of the activity tensor uses a matrix representing a correspondence relationship between an approximation value of the activity tensor and an approximation value of the acceptability tensor.

18. The non-transitory computer-readable medium according to claim 8, wherein the set of operations further comprising:
learning the relationship between the activity tensor and the acceptability tensor by using deep learning.
